(19) United States
(12) Patent Application Publication
(10) Pub. No.: US 2003/0163092 A1
(43) Pub. Date: Aug. 28, 2003

(54) Hypodermic syringe

(76) Inventors: David William Parker, Lancashire (GB); Colin Hamilton Burgess, Lancashire (GB)

Correspondence Address: NIXON & VANDERHYE, PC, 1100 N GLEBE ROAD, 8TH FLOOR, ARLINGTON, VA, 22201-4714, US

(21) Appl. No.: 10/239,640

(22) PCT Filed: Mar. 20, 2001
(86) PCT No.: PCT/GB01/01228

(30) Foreign Application Priority Data
Mar. 24, 2000 (GB) 0007269.4

(51) Int. Cl. A61M 5/32

(52) U.S. Cl. 604/198

(57) ABSTRACT

A syringe has a capsule assembly (1) which is pre-loaded with injectant fluid (1.10) and is provided with a needle (1.2) that is covered by a sealed needle sheath (1.4). The capsule assembly (1) is placed into a housing (2). The sealed capsule is prepared for use by removal of the sealed needle sheath (1.4). A plunger (4) with an associated piston (5) is used to input the fluid (1.10). The capsule assembly (1) including the needle (1.2) is automatically retracted into the housing (2) after use.

### FIELD OF THE INVENTION

This invention relates to hypodermic syringes with a retractable needle.

### BACKGROUND

Over recent years, the use of disposable syringes and needles has become increasing dangerous. Although the risk of an accidental scratch or puncture by a used needle, known as a needle stick injury, has always existed, the increased risk of infection with, for example HIV or hepatitis has become a growing concern to those involved in the use of disposable hypodermic syringes.

It is estimated that in the USA, there are approximately 1,000,000 needle stick injuries annually which result in some 20,000 incidences of infection of infection with HIV or hepatitis. These cause a considerable loss of life and the consequent cost is estimated at US$3 billion per annum currently.

With a view to overcoming this difficulty, a number of designs of hypodermic syringes with retractable needles have been proposed. One such design is known from U.S. Pat. No. 5,211,628 which discloses a hypodermic syringe including a housing, a plunger, a needle carrier with a needle mounted thereto positionable within the housing with the needle extending therefrom, a sheath mounted to the needle carrier and surrounding the needle, and a spring, the plunger and the spring configured such that when the syringe is used and the plunger reaches the needle carrier it becomes attached thereto and stored energy in the spring is released to retract the needle carrier into the housing.

Similar hypodermic syringes having an arrangement for retracting the needle into the plunger after use are known from U.S. Pat. No. 5,324,265, EP-A-0 505 300 and WO91/03269A.

All of the prior syringes mentioned above require the user to fill the syringe by placing the needle in a vial or other container containing injectant fluid and drawing the injectant fluid into the syringe. Thus, the syringe is charged by the operator immediately prior to use. However, approximately 45% of injections are currently given using a pre-loaded syringe which is typically smaller in capacity than operator loaded syringes. It is expected that the use of pre-loaded syringes will increase significantly with time.

Thus, there is a need to provide a pre-loaded syringe with a retractable needle assembly.

### SUMMARY OF THE INVENTION

In accordance with the invention, there is provided a syringe in which a needle is retracted by stored energy on completion of injection characterised in that the injectant is contained within a sealed needle and capsule assembly that includes a needle sheath wherein for use of the syringe, the seal is broken by removal of the sheath immediately prior to use.

Thus, in accordance with the invention, a syringe is provided which may be precharged with injectant, and for which the needle is automatically withdrawn after use to avoid the risk of needle stick injury.

### BRIEF DESCRIPTION OF THE DRAWINGS

In order that the invention may be more fully understood an embodiment thereof will now be described byway of example with reference to the accompanying drawings in which:

FIG. 1 is a schematic cross sectional view of an embodiment of syringe in accordance with the invention prior to use;

FIG. 2 is a schematic cross section of the syringe shown in FIG. 1, after use, with the needle withdrawn into the barrel of the device;

FIG. 3 is a schematic cross section of a first alternative embodiment of the invention;

FIG. 4 is a schematic cross section of a second alternative embodiment according to the invention;

FIG. 5A is a schematic perspective view of the capsule assembly, including the needle and needle sheath; and

FIG. 5B is a schematic illustration of an alternative needle capsule assembly.

### DETAILED DESCRIPTION

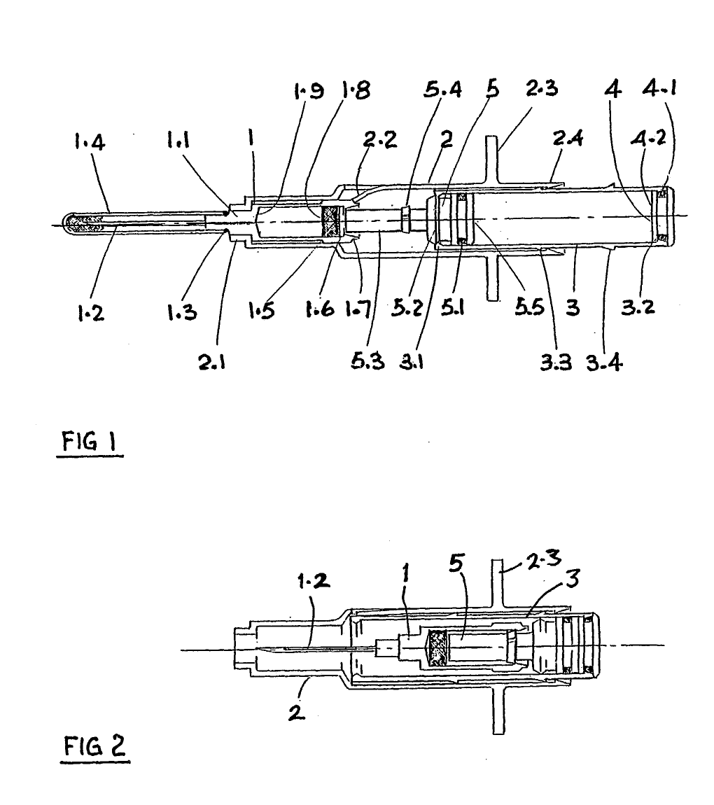

FIG. 1 illustrates an embodiment of syringe in accordance with the invention which comprises a capsule assembly 1 which is pre-charged with injectant and include the needle assembly with a needle sheath as will be described in more detail hereinafter. The capsule assembly is mounted in a cylindrical barrel 2 in which is mounted a plunger 3 provided with a closure piece 4 and a piston 5.

The capsule 1 containing injectant is integral with a needle mounting 1.1 and a needle 1.2. The needle mounting 1.1 contains a frangible sealant 1.3 to give a positive retention for a needle sheath 1.4.

The capsule assembly is of a diameter to fit through an opening 2.1 in the barrel 2 that has a region 1.5 of increased diameter in order to locate the capsule in place. The open end of the capsule 1 has an internal lip 1.6 and an external lip 1.7 and is provided with a resilient plunger disc 1.8. The needle end of the capsule 1.9 is dished. Injectant 1.10 is pre-charged into the capsule assembly 1 between the disc 1.8 and the dished end. 0.9.

The barrel 2 has a nose 2.1, the aperture of which is larger than the diameter of the needle sheath 1.4. One or more arms 2.2, which may be part of the barrel 2, are positioned so that they abut against the internal lip 1.7 of the capsule assembly. The barrel 2 has a finger plate 2.3 and an inwardly facing wedge sectioned ring 2.4. The nose 2.1 of the barrel and the needle mounting 1.1 may be non-circular as will be described hereinafter with reference to FIG. 5, in order to prevent relative axial rotation.

The plunger 3 has an inwardly facing triangular sectioned ring 3.1, an inward facing wedge shaped ring 3.2 and outwardly facing wedge sectioned rings 3.3 and 3.4. The closure piece 4 has a seal 4.1 which is held in place by a wedge section ring 3.2 of plunger 3.

The piston 5 carries the seal 5.1 which co-acts with the inner surface of the plunger 3 and a triangular section ring 5.2. The piston 5 includes a probe 5.3 which carries a resilient slit ring 5.4. The ring 5.4 is positioned so that it snaps past internal lip 1.6 before the injection stroke is completed.

During assembly, the closure piece 4 and the piston 5 are temporarily held in contact by the use of an appropriate lubricant between the two mating surfaces 4.2 and 5.5. These are inserted as one piece at the right-hand end of the plunger 3 shown in FIG. 1. The piston 5 is then drawn through the interior of the plunger 3 until the triangular sectioned ring 5.2 snaps past the inward facing triangular ring 3.1, creating a vacuum inside the plunger 3 and holding the piston 5 and the plunger 3 together as one piece. The vacuum is maintained by seals 4.1 and 5.1. The closure piece 4 is thus held at the end of the plunger 3.

Considering the capsule assembly 1, it is pre-charged with injectant fluid between the dished needle end 1.9 and the resilient plunger disc 1.8. Also, the assembly is fitted with the needle sheath 1.4 over the needle 1.2, which is held in place by the frangible sealant 1.3.

The capsule assembly 1 is passed through the interior of the barrel 2 until the needle mounting 1.1 rests in the nose 2.1 of the barrel. The aperture 2.1 is sufficiently large to allow the passage of the needle 1.2 and the protective, sealed sheath 1.4 through the aperture. The sheath 1.4 affords protection to the needle 1.2 and guidance in entering and locating the capsule assembly in the nose 2.1 of the barrel. The larger size of the nose aperture 2.1 in relation to the diameter of the needle 1.2 enables a bent needle to be retracted as will be evident hereinafter. The or each arm 2.2 abuts against the external lip 1.7 thereby restraining the capsule assembly 1 from inward movement. The seal afforded by the sheath 1.4 prevents inward movement of the plunger 3 being made before the sheath 1.4 has been removed from the needle 1.2.

The plunger 3 and the piston 5 are inserted into the barrel 2 as one piece until wedge sectioned ring 3.3 snaps past wedge sectioned ring 2.4. It is not possible thereafter for the plunger to be withdrawn from the barrel.

The syringe is thus assembled ready for use.

When it is desired to use the syringe, the sheath 1.4 is removed from the needle 1.2. The non-circular construction of the needle mounting 1.1 and the nose 2.1 allows the sheath 1.4 to be rotated independently of its associated components, thereby breaking the seal between them and facilitating removal of the sheath 1.4. The syringe can then be operated according to established practice for disposable syringes. Thus, the syringe is gripped by the user such that the plunger 3 is slidably inserted into the barrel 2, with the user gripping the finger plate 2.3. On completion of the injection stroke, automatic needle retraction is triggered by further pressure on the plunger 3, as will now be described in detail.

Pressure on the plunger 3 moves the plunger and the piston 5 together as a unit through the interior-of the barrel 2 towards the capsule assembly 1. As the probe 5.3 enters the open end of the capsule 1, it forces the sealing disc 1.8 towards the needle end of the capsule thereby expelling the injectant 1.10 through the needle 1.2. As the disc 1.8 reaches the closed end 1.9 of the capsule, the resilient split ring 5.4 is compressed so as to move past the internal lip 1.6 and the plunger 3 lifts arm 2.2 away from the internal lip 1.7. Final inward movement of the probe 5.3 distorts the disc 1.8 against the dished end 1.9 of the capsule 1, thereby ensuring complete evacuation of the injectant. When the piston 5 reaches the end of the barrel 2, further inward movement of the piston 5 is prevented so that further inward pressure on the plunger 3 snaps the triangular ring 3.1 past the wedge sectioned ring 5.2, thereby releasing the piston 5 and allowing the vacuum within the plunger 3 to draw the piston 5 and the capsule assembly 1 into the plunger 3.

As this retraction commences, the pressure exerted by the probe 5.3 on the disc 1.8 is released. This allows the disc 1.8 to revert to its normal shape and in doing so, residual injectant in the needle 1.2 is drawn into the resultant cavities thereby preventing any seepage of residual injectant from the needle 1.2.

Towards the end of the injection stroke, and before retraction has commenced, lip 3.4 snaps past wedge section ring 2.4 thus locking the plunger 3 with the barrel 2 and preventing the plunger from being withdrawn therefrom.

FIG. 2 illustrates the resulting configuration of the syringe component shown in FIG. 1, after needle retraction has occurred. It will be seen that the needle 1.2 has been withdrawn completely within the barrel 2 thereby obviating the risk of needle stick injury.

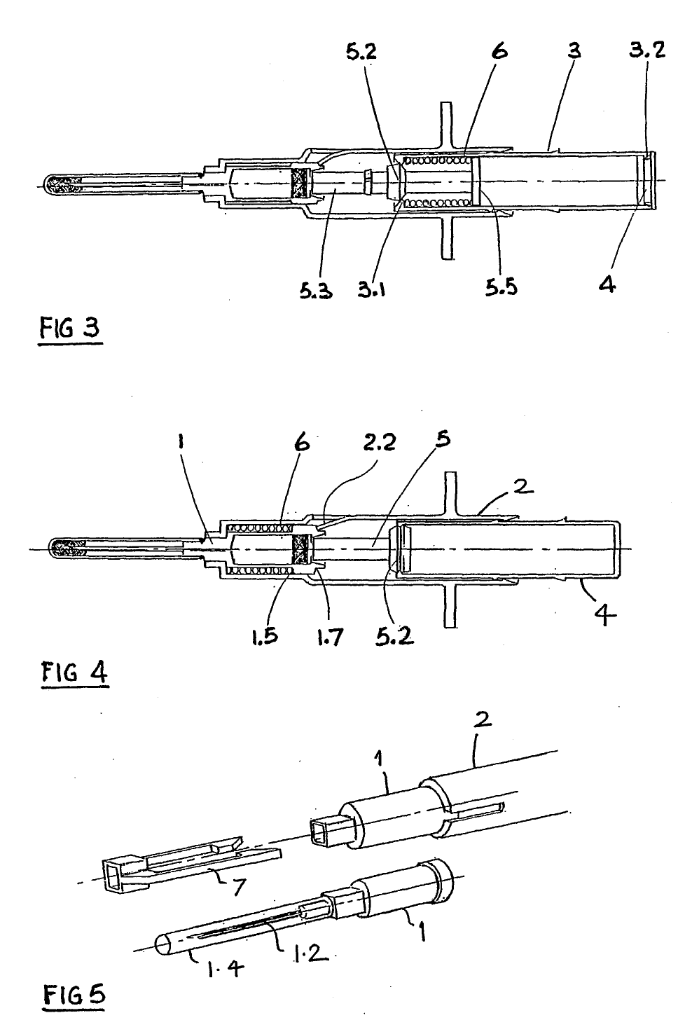

FIG. 3 illustrates an alternative construction in which the energy used to effect retraction is provided by compression spring 6 rather than the vacuum between the plunger 3 and piston 5, of the embodiment of FIG. 1. In the embodiment of FIG. 3, the spring 6 is located within the plunger 3 and is of the maximum diameter allowed by the internal dimensions of the plunger 3. The triangular section ring 3.1 provides a stop which locates the spring 6. The probe 5.3 is passed through the interior of the spring 6 and inward pressure is applied to face 5.5, moving the piston 5 through the plunger 3 simultaneously compressing the spring 6 until the triangular sectioned ring 5.2 snaps past the triangular sectioned ring 3.1. The spring 6 is now held in compression and the plunger 3 and piston 5 are held together as one piece. The closure piece 4 is now fitted into the barrel and held by the wedge sectioned ring 3.2. Operation of the device is as previously described with reference to FIG. 1, with the retraction of the needle being performed under the force of compression spring 6 rather than the vacuum as previously described.

FIG. 4 illustrates a configuration in which the compression spring 6 is located within the barrel 2, so as to surround the capsule assembly 1. The capsule assembly 1 is passed through the interior of the spring 6 which is compressed against the locally increased diameter 1.5 as the capsule assembly 1 is loaded into place. The capsule assembly 1 is held in position and the spring 6 is held in compression by the or each arm 2.2. acting on the external lip 1.7. The piston 5 has a triangularly shaped groove 5.2. In the embodiment of FIG. 4, assembly is achieved as previously described, except that the piston 5 and the plunger 3 are joined by pushing the piston 5 into the plunger 3 until the triangularly shaped groove 5.2 is retained by the triangular section ring 3.1. Operation of the embodiment of FIG. 4 is carried out as previously described except that when the final inward pressure on the plunger 3 snaps the triangular section ring 3.1 out of the groove 5.2, the spring 6 forces the capsule assembly 1 and the piston 5 into the plunger 3.

FIG. 5A illustrates in perspective the capsule assembly 1 including the needle 1.2 and needle sheath 1.4 with its rectangular i.e. non-cylindrical nose portion 1.1 which co-acts with the close fitting, similarly shaped aperture in the barrel 2 (not shown in FIG. 5). Thus, rotation of the needle and capsule assembly 1 is prevented by the co-acting non-circular faces of the assembly 1 and barrel aperture 2.1 when the needle sheath is twisted so as to break the seal 1.3 and facilitate removal of the sheath. It will be understood that other means of achieving this may be used such as an off-set pin and socket or other means of producing non-rotary engagement between the capsule assembly 1 and the barrel 2.

FIG. 5B illustrates an alternative configuration in which the capsule assembly 1 can be retained in the housing 2 before retraction of the needle is initiated, using a clip 7.

The described embodiments of the invention thus provide for safe and reliable operation of a pre-loaded syringe and have the advantage that the capsule assembly 1 containing the injectant 1.10 is pre-loaded and sealed under controlled conditions ready for incorporation into the syringe. Furthermore the injectant and the quantity thereof may be identified on the capsule, for example by means of a barcode which may be magnetic and may provide data for inclusion into the patients record, stock control and quality control purposes. Additionally, the user is not required to expel excess air from the needle prior to use and no calibrations for the dose are necessary because the dose is pre-set by the volume of injectant included into the capsule assembly 1. It will be understood that partial rotation of the needle sheath 1.4 breaks the seal and facilitates removal of the needle sheath from the needle 1.4 immediately prior to use. Seepage and spillage of the injectant are prevented as previously described.

Thus, the syringe according to the invention is reliable, instinctive in its operation and capable of being used with one hand. Furthermore, the needle is automatically retracted completely following the injection and, because the aperture 2.1 in the nose of barrel is relatively large, it has the capacity to retract a bent needle. Furthermore, re-exposure of the needle cannot occur after the injection thereby minimising the risk of a needle stick injury. Also, accidental retraction of the needle before the injection is prevented.

The described examples of syringe according to the invention are so configured that there are locked closed after use to provide for compact and safe disposal. The described syringes can be manufactured at low cost.

1. A syringe including a needle retractable by stored energy on completion injection, wherein an injectant is contained within a sealed needle and capsule assembly having a needle sheath covering the needle, in which the seal is broken by removal of the needle sheath immediately before use.

2. A syringe as claimed in claim 1. in which only the needle and capsule assembly contact the injectant.

3. A syringe as claimed in claim 1. or claim 2. in which the needle sheath is provided with coding indicating injectant constituents and quantity.

4. A syringe as claimed in claim 3 in which the coding is by bar code.

5. A syringe as claimed in claim 3 in which the coding is magnetic.

6. A syringe as claimed in claim 3 in which the coding provides data for patients' records, stock control and quality control purposes.

7. A syringe as claimed in any preceding claim in which the stored energy is provided by a vacuum.

8. A syringe as claimed in claims I to 6 in which the stored energy is contained in a spring.

9. A syringe as claimed in claim 8 including a plunger, wherein the spring is maintained in compression and located within the plunger.

10. A syringe as claimed in claim 8 in which the spring is maintained in compression and located around the capsule assembly.

11. A syringe as claimed in claims 7 and 9 in which the retractable part of the plunger couples to the capsule and needle assembly near completion of the injection stroke.

12. A syringe as claimed in any preceding claim in which the plunger is axially collapsible at a set overload.

13. A syringe as claimed in any preceding claim in which the needle and capsule assembly is retained prior to retraction by a catch displaceable by the plunger on completion of the injection stroke.

14. A syringe as claimed in claim 13 in which the said catch is an integrally formed feature of the housing.

15. A syringe as claimed in claim 13 in which the said catch is a separate component or assembly.

16. A syringe as claimed in any preceding claim in which the needle and capsule assembly can be installed through the main housing of the syringe.

17. A syringe as claimed in any preceding claim in which the needle end of the housing is provided with a non cylindrical aperture through which the needle and sheath can pass and into which a co-acting non cylindrical section of the capsule enters to allow a torque to be applied to the sheath to break the seal and facilitate its removal.

18. A syringe as claimed in claim 17 in which the clearance provided by the non cylindrical aperture in the housing allows he stored energy to retract and retain within the housing a bent needle.

19. A syringe as claimed in claims 1 to 16 in which the needle end of the housing is provided with a cylindrical aperture through which the needle and sheath can pass and into which a co-acting cylindrical section of the capsule enters, relative rotational movement being prevented by co-acting protuberance and receptor on the two parts, to allow a torque to be applied to the sheath to break the seal and facilitate its removal.

20. A syringe as claimed in claim 19. in which the clearance by the cylindrical aperture in he housing allows the stored energy to retract and retain within the housing a bent needle.

21. A syringe as claimed in any preceding claim in which the needle and capsule assembly contains an axially moveable resilient disc which under inward pressure will expel the injectant.

22. A syringe as claimed in claim 21 in which the resilience of the disc allows distortion of its natural shape under pressure of the injection stroke to replicate the geometry of the needle end of the capsule to provide complete evacuation of the capsule.

23. A syringe as claimed in claim 21 in which the resilience of the disc allows return to its natural shape after the pressure of the injection stroke is released and thereby drawing in any residual injectant from the needle bore.
